(19) United States
(12) Patent Application Publication
(10) Pub. No.: US 2024/0327321 A1
(43) Pub. Date: Oct. 3, 2024

(54) HARDMASK COMPOSITION, HARDMASK LAYER AND METHOD OF FORMING PATTERNS

(71) Applicant: SAMSUNG SDI CO., LTD., Yongin-si (KR)

(72) Inventor: Seil CHOI, Suwon-si (KR)

(21) Appl. No.: 18/417,298

(22) Filed: Jan. 19, 2024

(30) Foreign Application Priority Data
Mar. 28, 2023 (KR) 10-2023-0040766

(51) Int. Cl. C07C 39/17; G03F 7/09; G03F 7/26

(52) U.S. Cl. CPC C07C 39/17; G03F 7/094; G03F 7/265

(57) ABSTRACT

A hardmask composition, including a hardmask layer including a cured product of the hardmask composition, and a method of forming patterns using the hardmask layer including a cured product of the hardmask composition, the hardmask composition including a compound represented by Chemical Formula 1; and a solvent.

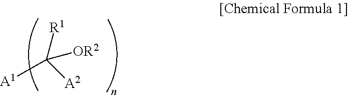

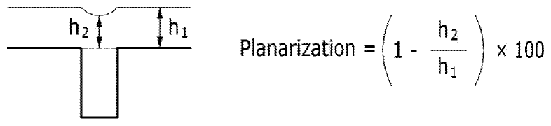

### CROSS-REFERENCE TO RELATED APPLICATION

This application claims priority to and the benefit of Korean Patent Application No. 10-2023-0040766 filed in the Korean Intellectual Property Office on Mar. 28, 2023, the entire contents of which are incorporated herein by reference.

### BACKGROUND

### 1. Field

Embodiments relate to a hardmask composition, a hardmask layer, and a method of forming patterns.

### 2. Description of the Related Art

Recently, the semiconductor industry has developed to an ultra-fine technique having a pattern of several to several tens nanometer size. Such ultrafine technique may utilize lithographic techniques.

Some lithographic techniques may include providing a material layer on a semiconductor substrate; coating a photoresist layer thereon; exposing and developing the same to provide a photoresist pattern; and etching a material layer using the photoresist pattern as a mask.

According to small-sizing the pattern to be formed, it may be difficult to provide a fine pattern having an excellent profile by only using some lithographic techniques. Accordingly, an auxiliary layer, called a hardmask layer, may be formed between the material layer and the photoresist layer to provide a fine pattern.

### SUMMARY

The embodiments may be realized by providing a hardmask composition including a compound represented by Chemical Formula 1; and a solvent,

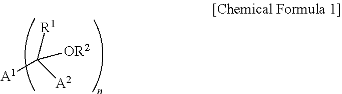

wherein, in Chemical Formula 1, A¹ and A² are each independently a substituted or unsubstituted C6 to C30 aromatic hydrocarbon group, or a group in which substituted or unsubstituted C6 to C30 aromatic hydrocarbon groups are linked by a single bond, a substituted or unsubstituted C1 to C5 alkylene group, or a combination thereof,
R¹ is a substituted or unsubstituted monovalent C1 to C30 saturated aliphatic hydrocarbon group, a substituted or unsubstituted monovalent C2 to C30 unsaturated aliphatic hydrocarbon group, a substituted or unsubstituted monovalent C3 to C30 saturated or unsaturated alicyclic hydrocarbon group, or a substituted or unsubstituted C6 to C30 aromatic hydrocarbon group,
R² is hydrogen, deuterium, or a substituted or unsubstituted C1 to C20 alkyl group,
n is an integer of 1 to 6, and
if n is an integer of 2 or more, each of R¹, R², and A² is the same or different from each other.

A¹ and A² in Chemical Formula 1 may each independently be a substituted or unsubstituted group of a moiety of Group 1:

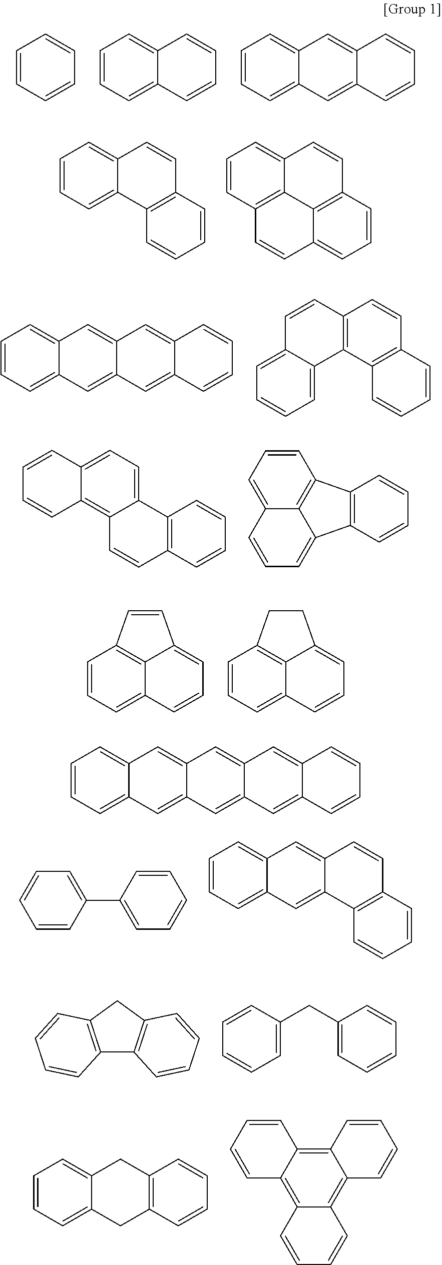

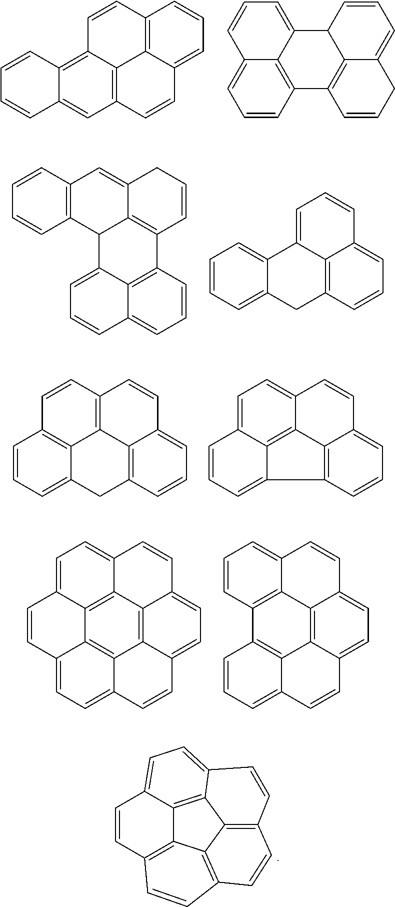

A¹ and A² in Chemical Formula 1 may each independently be, a substituted or unsubstituted group of a moiety of Group 1-1:

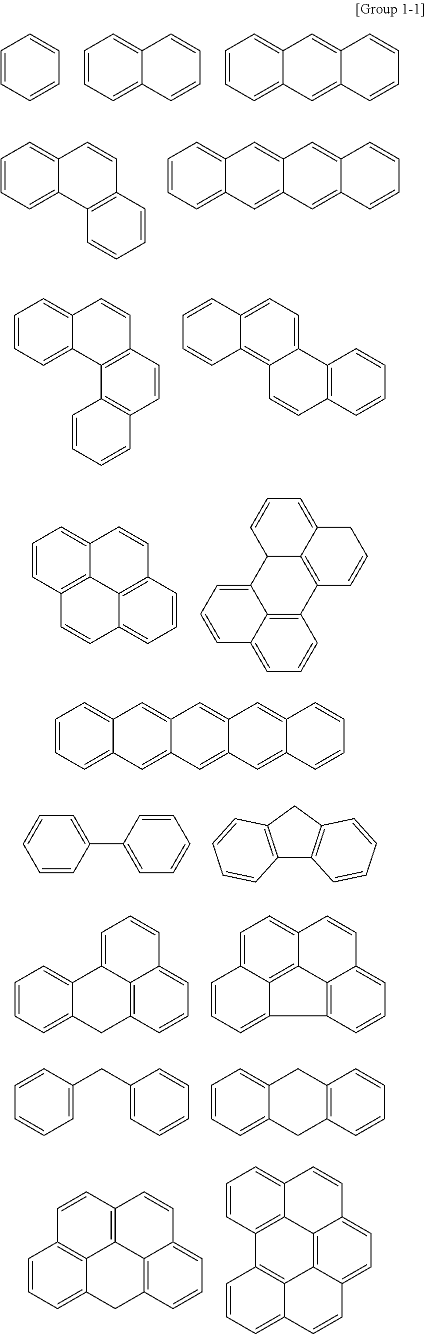

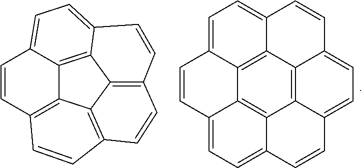

A¹ in Chemical Formula 1 may be any one of a substituted or unsubstituted group of a moiety of Group 1-2:

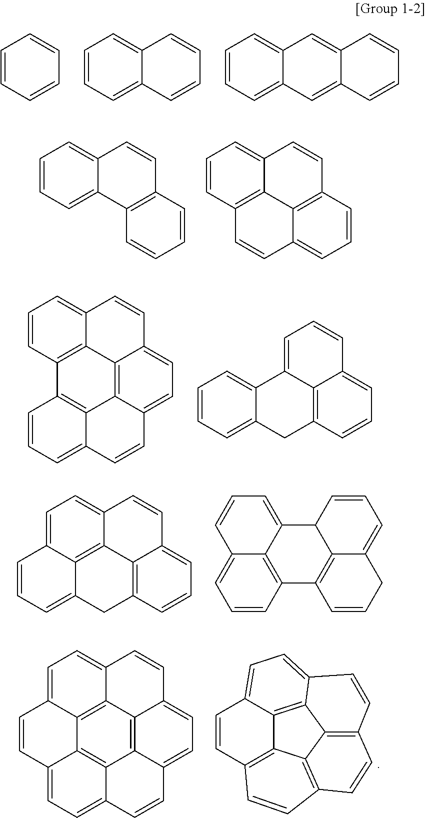

A² in Chemical Formula 1 may be any substituted or unsubstituted group of a moiety of Group 1-3:

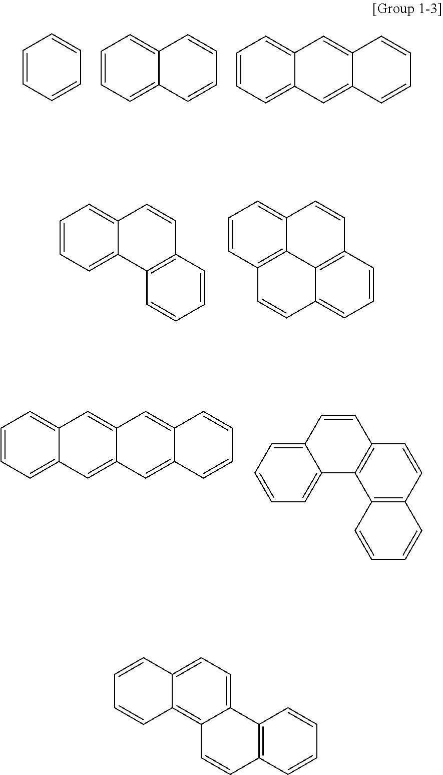

R¹ in Chemical Formula 1 may be a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group.

R² in Chemical Formula 1 may be hydrogen, deuterium, a substituted or unsubstituted methyl group, a substituted or unsubstituted ethyl group, a substituted or unsubstituted propyl group, a substituted or unsubstituted butyl group, or a substituted or unsubstituted pentyl group.

In Chemical Formula 1, R¹ may be a substituted or unsubstituted C1 to C20 alkyl group, or a substituted or unsubstituted C6 to C20 aryl group, R² may be hydrogen, deuterium, a substituted or unsubstituted methyl group, or a substituted or unsubstituted ethyl group, and n may be an integer of 1 to 3.

In Chemical Formula 1, n may be 2 or 3, and R¹, R², and A² may all be the same.

In Chemical Formula 1, R¹ may be a substituted or unsubstituted C1 to C10 alkyl group, R² may be hydrogen, or deuterium, and n may be 2.

The compound represented by Chemical Formula 1 may be represented by one of Chemical Formula 2 to Chemical Formula 4:

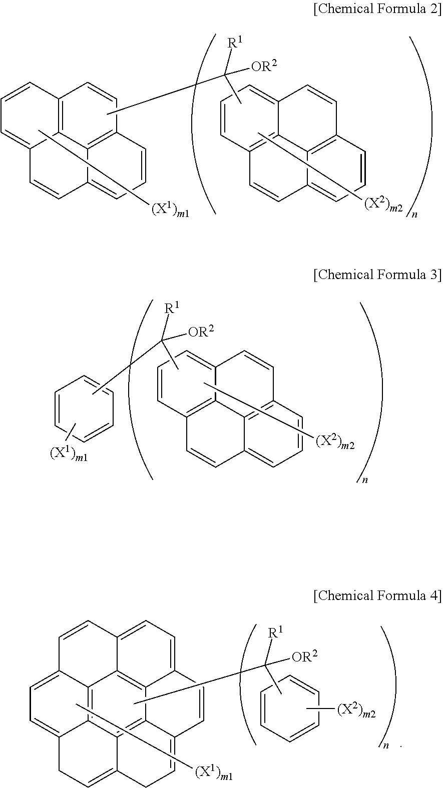

In Chemical Formula 2 to Chemical Formula 4, R¹ may be a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group,
R² may be a hydrogen, deuterium, or a substituted or unsubstituted C1 to C10 alkyl group,
X¹ and X² may each independently be, deuterium, a hydroxy group, a halogen atom, a thiol group, a substituted or unsubstituted C1 to C10 alkoxy group, or a substituted or unsubstituted C1 to C10 alkyl group,
n may be an integer of 1 to 3, and
m1 and m2 may each independently be an integer of 0 to 5.

The compound represented by Chemical Formula 1 may be represented by any one of Chemical Formula 5 to Chemical Formula 10:

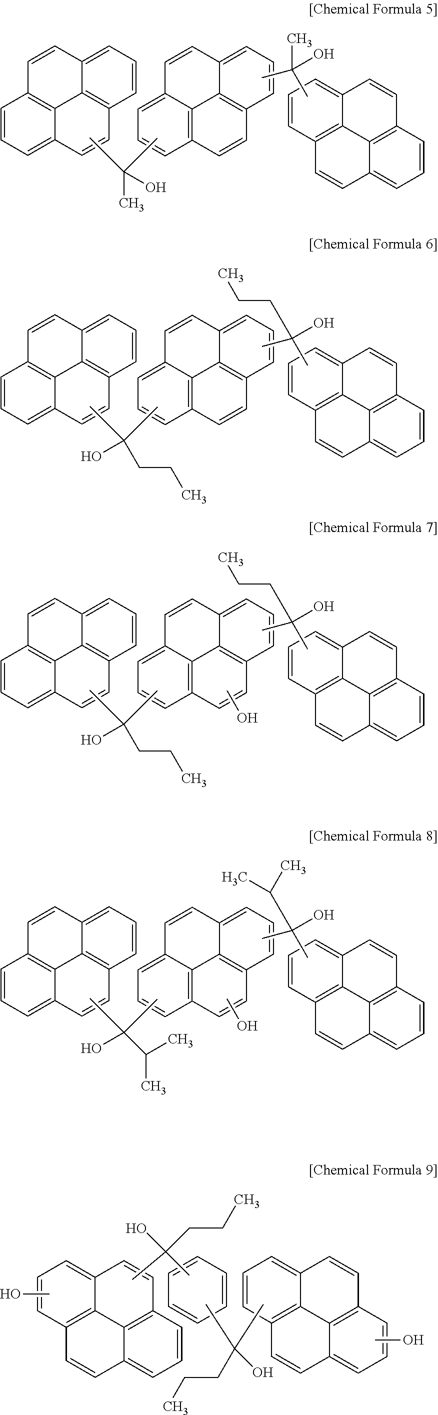

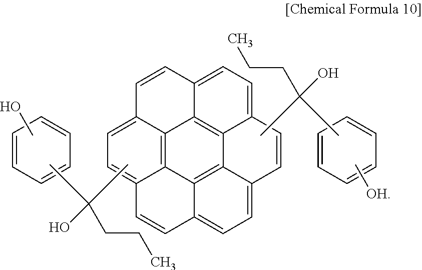

The compound may have a molecular weight of about 500 g/mol to about 10,000 g/mol.

The compound may be included in an amount of about 0.1 wt % to about 30 wt %, based on a total weight of the hardmask composition.

The solvent may include propylene glycol, propylene glycol diacetate, methoxy propanediol, diethylene glycol, diethylene glycol butylether, tri (ethylene glycol) monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, methylpyrrolidinone, acetylacetone, or ethyl 3-ethoxypropionate.

The embodiments may be realized by providing a hardmask layer including a cured product of the hardmask composition according to an embodiment.

The embodiments may be realized by providing a method of forming patterns the method including providing a material layer on a substrate; applying the hardmask composition according to an embodiment to the material layer to form a hardmask layer; heat-treating the hardmask composition to form a hardmask layer; forming a photoresist layer on the hardmask layer, exposing and developing the photoresist layer to form a photoresist pattern; selectively removing the hardmask layer using the photoresist pattern to expose a portion of the material layer; and etching an exposed part of the material layer.

Forming the hardmask layer may include heat-treating at about 100° C. to about 1,000° C.

### BRIEF DESCRIPTION OF THE DRAWING

Features will become apparent to those of skill in the art by describing in detail exemplary embodiments with reference to the attached drawing in which:

the FIGURE shows a reference diagram illustrating a level difference of a hardmask layer to explain a method of evaluating planarization characteristics and a calculation formula for evaluating planarization characteristics.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, ‘substituted’ may refer to replacement of a hydrogen atom of a compound by a substituent selected from a halogen atom (F, Br, Cl, or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a vinyl group, a C1 to C20 alkyl group, a C2 to C20 alkenyl group, a C2 to C20 alkynyl group, a C6 to C30 aryl group, a C7 to C30 arylalkyl group, a C9 to C30 allylaryl group, a C1 to C30 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, or a combination thereof. As used herein, hydrogen substitution (—H) may include deuterium substitution (-D) or tritium substitution (-T). For example, any hydrogen in any compound described herein may be protium, deuterium, or tritium (e.g., based on natural or artificial substitution). As used herein, the term “or” is not necessarily an exclusive term, e.g., “A or B” would include A, B, or A and B.

In addition, adjacent two substituents of the substituted halogen atom (F, Br, Cl, or I), the hydroxy group, the nitro group, the cyano group, the amino group, the azido group, the amidino group, the hydrazino group, the hydrazono group, the carbonyl group, the carbamyl group, the thiol group, the ester group, the carboxyl group or the salt thereof, the sulfonic acid group or the salt thereof, the phosphoric acid or the salt thereof, the C1 to C30 alkyl group, the C2 to C30 alkenyl group, the C2 to C30 alkynyl group, the C6 to C30 aryl group, the C7 to C30 arylalkyl group, the C1 to C30 alkoxy group, the C1 to C20 heteroalkyl group, the C3 to C20 heteroarylalkyl group, the C3 to C30 cycloalkyl group, the C3 to C15 cycloalkenyl group, the C6 to C15 cycloalkynyl group, the C2 to C30 heterocyclic group may be fused to form a ring.

As used herein, when a definition is not otherwise provided, “aromatic hydrocarbon” refers to a group having one or more hydrocarbon aromatic moieties, including a form in which hydrocarbon aromatic moieties are linked by a single bond, a non-aromatic fused ring form in which hydrocarbon aromatic moieties are fused directly or indirectly, or a combination thereof as well as non-fused aromatic hydrocarbon rings, fused aromatic hydrocarbon rings.

More specifically, the substituted or unsubstituted aromatic hydrocarbon group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted terphenyl group, a substituted or unsubstituted quaterphenyl group, a substituted or unsubstituted chrysenyl group, a substituted or unsubstituted triphenylenyl group, a substituted or unsubstituted perylenyl group, a substituted or unsubstituted indenyl group, a combination thereof, or a combined fused ring of the foregoing groups.

As used herein, the polymer may include both an oligomer and a polymer.

Unless otherwise specified in the present specification, the “molecular weight” is measured by dissolving a powder sample in tetrahydrofuran (THF) and then using 1200 series Gel Permeation Chromatography (GPC) of Agilent Technologies (column is Shodex Company LF-804, standard sample is Shodex company polystyrene).

The hardmask composition according to some embodiments may include, e.g., a compound represented by Chemical Formula 1, and a solvent.

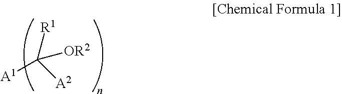

In Chemical Formula 1, A¹ and A² may each independently be or include, e.g., a substituted or unsubstituted C6 to C30 aromatic hydrocarbon group, or a group in which substituted or unsubstituted C6 to C30 aromatic hydrocarbon groups may be linked by a single bond, a substituted or unsubstituted C1 to C5 alkylene group, or a combination thereof.

R¹ may be or include, e.g., a substituted or unsubstituted monovalent C1 to C30 saturated aliphatic hydrocarbon group, a substituted or unsubstituted monovalent C2 to C30 unsaturated aliphatic hydrocarbon group, a substituted or unsubstituted monovalent C3 to C30 saturated or unsaturated alicyclic hydrocarbon group, or a substituted or unsubstituted C6 to C30 aromatic hydrocarbon group.

R² may be or include, e.g., hydrogen, deuterium, or a substituted or unsubstituted C1 to C20 alkyl group.

n may be an integer of 1 to 6.

In an implementation, if n is an integer of 2 or more, each of R¹, R², and A² may be the same or different from each other.

As shown in Chemical Formula 1, the compound according to an implementation may include a quaternary carbon, and an aromatic hydrocarbon group, a saturated or unsaturated aliphatic hydrocarbon group, or a saturated or unsaturated alicyclic hydrocarbon group, or the like directly linked to the quaternary carbon, thereby increasing a carbon content in the compound, and thus increasing the carbon content in the hardmask composition and helping improve etch resistance of the hardmask layer formed therefrom.

In addition, the compound according to an implementation may include a quaternary carbon, and a hydroxy group or an alkoxy group may be linked to the quaternary carbon, so that solubility of the compound in a solvent may be improved, and a hardmask composition containing this may be effectively applied to a spin-on coating process. In addition, the hydroxy group or alkoxy group may help crosslinking between compounds, and thus a composition containing the compound may exhibit excellent crosslinking characteristics. In addition, since the compound may be crosslinked into a high molecular weight polymer form within a short period of time during heat treatment, the hardmask layer formed therefrom may have excellent heat resistance and excellent etch resistance.

In an implementation, A¹ and A² of Chemical Formula 1 may each independently be, e.g., a substituted or unsubstituted group of a moiety of Group 1.

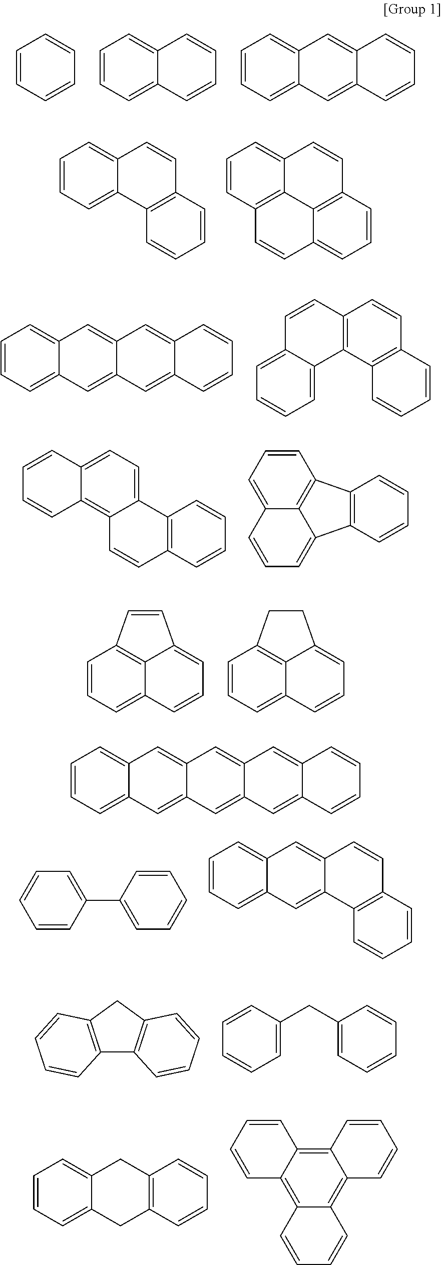

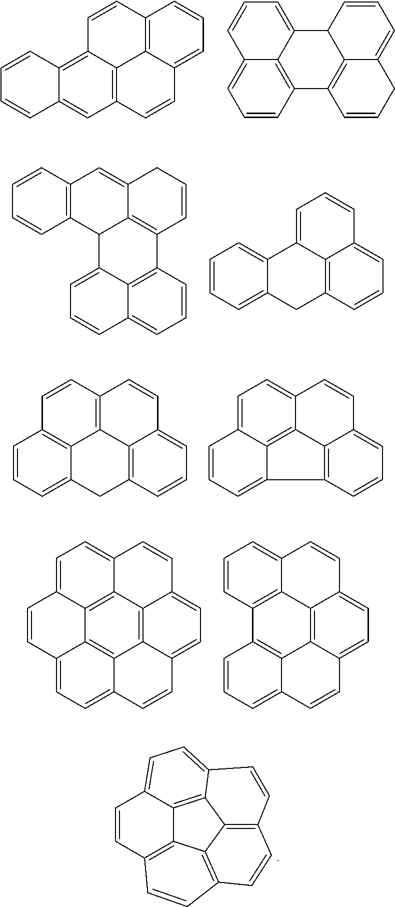

In an implementation, A¹ and A³ in Chemical Formula 1 may each independently be, e.g., a substituted or unsubstituted group of a moiety of Group 1-1.

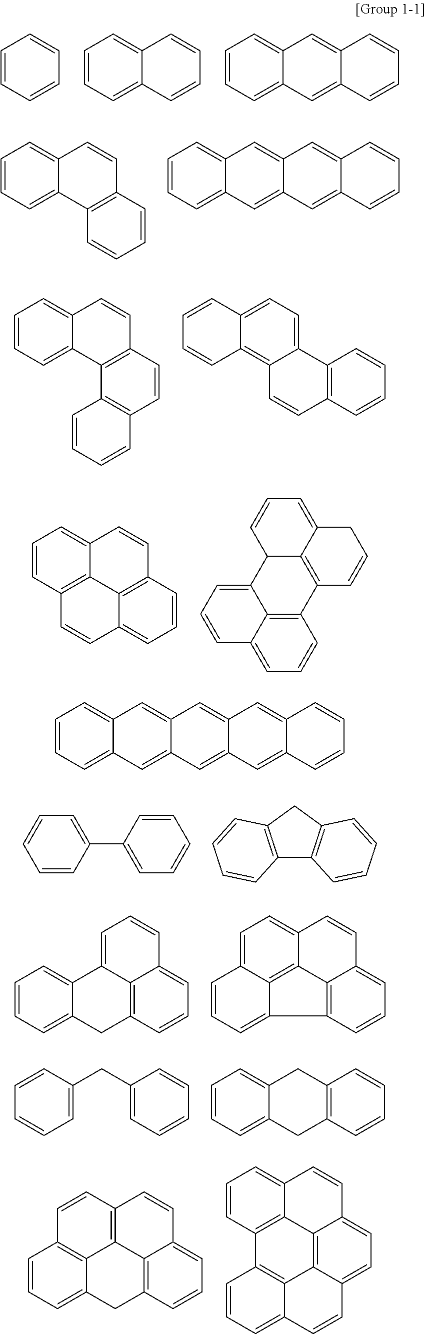

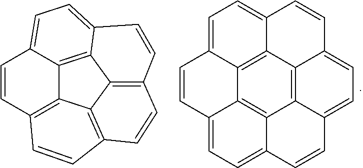

In an implementation, A¹ in Chemical Formula 1 may be, e.g., a substituted or unsubstituted group of a moiety of Group 1-2, such as, benzene, naphthalene, anthracene, phenanthrene, pyrene, or coronene. In an implementation, A¹ in Chemical Formula 1 may be, e.g., benzene, pyrene, or coronene.

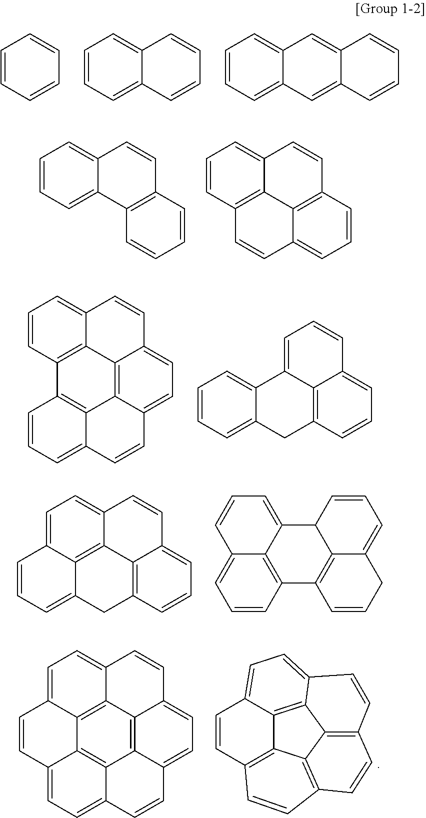

In an implementation, A² of Chemical Formula 1 may be, e.g., a substituted or unsubstituted group of a moiety of Group 1-3, such as, benzene, naphthalene, anthracene, phenanthrene, or pyrene. In an implementation, A² of Chemical Formula 1 may be, e.g., benzene or pyrene.

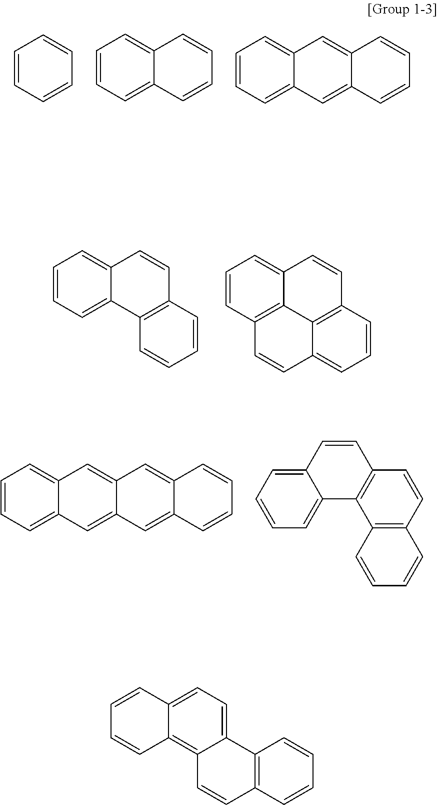

In an implementation, R¹ in Chemical Formula 1 may be, e.g., a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group. In an implementation, Chemical Formula 1 may be, e.g., a substituted or unsubstituted C1 to C20 alkyl group, or a substituted or unsubstituted C6 to C20 aryl group. In an implementation, Chemical Formula 1 may be, e.g., a substituted or unsubstituted C1 to C10 alkyl group.

In an implementation, if R¹ in Chemical Formula 1 has the substituents listed above rather than hydrogen, the carbon content of the compound according to some embodiments may be maximized.

R² in Chemical Formula 1 may be, e.g., hydrogen, deuterium, or a substituted or unsubstituted C1 to C10 alkyl group. In an implementation, R² in Chemical Formula 1 may be, e.g., hydrogen, deuterium, a substituted or unsubstituted methyl group, a substituted or unsubstituted ethyl group, a substituted or unsubstituted propyl group, a substituted or unsubstituted butyl group, or a substituted or unsubstituted pentyl group, e.g., hydrogen, deuterium, a substituted or unsubstituted methyl group, or a substituted or unsubstituted ethyl group. In an implementation, R² in Chemical Formula 1 may be, e.g., hydrogen or deuterium.

In Chemical Formula 1, n may be an integer of 1 to 6, e.g., an integer of 1 to 3. In an implementation, n may be 2 or 3, e.g., n may be 2. If n in Chemical Formula 1 is 2, the solubility of the compound according to some embodiments in a solvent may be optimized and etch resistance of a hardmask layer formed from a hardmask composition containing the compound may be maximized.

In an implementation, if n in Chemical Formula 1 is an integer of 2 or more, each of R¹, R², and A² may be the same or different from each other. In an implementation, if n is 2 or more, each of two or more R¹'s may be the same or different from each other, each of two or more R²s may be the same or different from each other, and each of two or more A²s may be the same or different from each other.

In an implementation, the compound represented by Chemical Formula 1 may be represented by, e.g., one of Chemical Formula 2 to Chemical Formula 4.

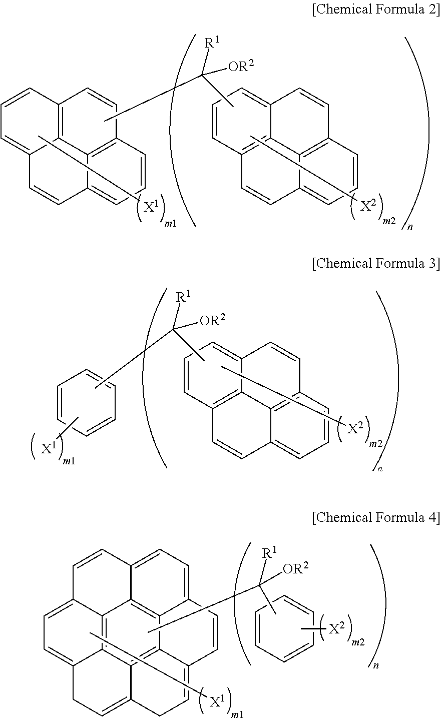

In Chemical Formula 2 to Chemical Formula 4, R¹ may be, e.g., a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group.

R² may be, e.g., hydrogen, deuterium, or a substituted or unsubstituted C1 to C10 alkyl group.

X¹ and X² may each independently be, e.g., deuterium, a hydroxy group, a halogen atom, a thiol group, a substituted or unsubstituted C1 to C10 alkoxy group, or a substituted or unsubstituted C1 to C10 alkyl group.

n may be an integer of 1 to 3.

m1 and m2 may each independently be an integer of 0 to 5.

In an implementation, the compound represented by Chemical Formula 1 may be represented by, e.g., one of Chemical Formula 5 to Chemical Formula 10.

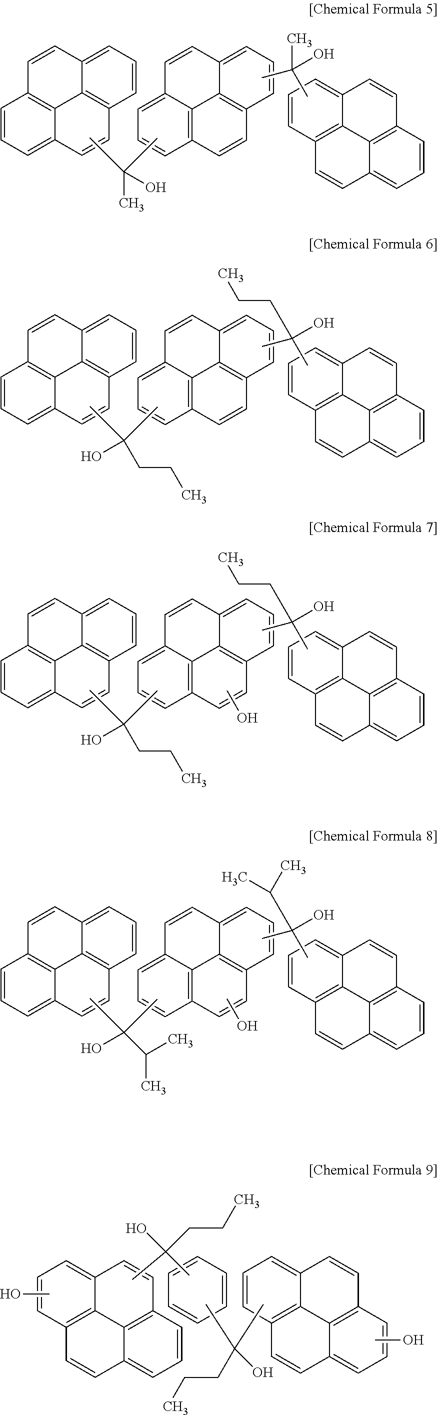

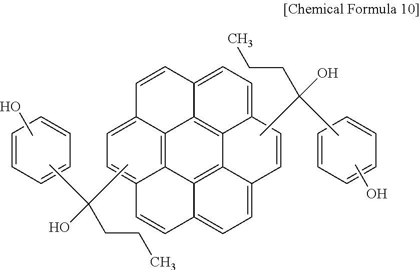

The compound may have a molecular weight of about, e.g., 500 g/mol to about 10,000 g/mol. In an implementation, the compound may have a molecular weight of about 500 g/mol to about 9,500 g/mol, about 500 g/mol to about 9,000 g/mol, about 600 g/mol to about 8,500 g/mol, about 600 g/mol to about 8,000 g/mol, about 700 g/mol to about 7,500 g/mol, about 700 g/mol to about 7,000 g/mol. By having a molecular weight within the above ranges, the carbon content and solubility in the solvent of the hardmask composition including the above compound may be adjusted and optimized.

The compound may be included in an amount of about 0.1 wt % to about 30 wt %, based on a total weight of the hardmask composition. In an implementation, the compound may be included in an amount of about 0.2 wt % to about 30 wt %, about 0.5 wt % to about 30 wt %, about 1 wt % to about 30 wt %, about 1 wt % to about 25 wt %, or about 1 wt % to about 20 wt %. By including the compound within the above ranges, a thickness, a surface roughness, and a planarization degree of the hardmask may be easily adjusted.

The hardmask composition may include a solvent. In an implementation, the solvent may include propylene glycol, propylene glycol diacetate, methoxy propanediol, diethylene glycol, diethylene glycol butyl ether, tri (ethylene glycol) monomethyl ether, propylene glycol monomethyl ether, propylene glycol monomethyl ether acetate, cyclohexanone, ethyl lactate, gamma-butyrolactone, N,N-dimethylformamide, N,N-dimethylacetamide, methylpyrrolidone, methylpyrrolidinone, acetylacetone, ethyl 3-ethoxypropionate, or the like. In an implementation, the solvent may include a suitable solvent that has sufficient solubility or dispersibility for the compound.

The hardmask composition may further include an additive, e.g., a surfactant, a crosslinking agent, a thermal acid generator, or a plasticizer.

The surfactant may include, e.g., a fluoroalkyl compound, alkylbenzenesulfonate, alkylpyridinium salt, polyethylene glycol, a quaternary ammonium salt, or the like.

The crosslinking agent may include, e.g., a melamine, a substituted urea, or a polymer crosslinking agent. In an implementation, it may be a crosslinking agent having at least two crosslinking substituents, e.g., methoxymethylated glycoruryl, butoxymethylated glycoruryl, methoxymethylated melamine, butoxymethylated melamine, methoxymethylated benzoguanamine, butoxy methylated benzoguanamine, methoxymethylated urea, butoxymethylated urea, methoxymethylated thiourea, or butoxymethylated thiourea.

In an implementation, as the crosslinking agent, a crosslinking agent having high heat resistance may be used. The crosslinking agent having high heat resistance may include a compound containing a crosslinking substituent having an aromatic ring (e.g., a benzene ring or a naphthalene ring) in the molecule.

The thermal acid generator may include, e.g., an acid compound, such as p-toluenesulfonic acid, trifluoromethanesulfonic acid, pyridinium p-toluenesulfonic acid, salicylic acid, sulfosalicylic acid, citric acid, benzoic acid, hydroxybenzoic acid, naphthalenecarboxylic acid or 2,4,4,6-tetrabromocyclohexadienone, benzointosylate, 2-nitrobenzyltosylate, and other organic sulfonic acid alkyl esters.

According to some embodiments, a hardmask layer including a cured product of the aforementioned hardmask composition may be provided.

Hereinafter, a method of forming patterns using the aforementioned hardmask composition is described.

A method of forming patterns according to some embodiments may include providing a material layer on a substrate, applying a hardmask composition including the aforementioned compound and solvent to the material layer, heat-treating the hardmask composition to form a hardmask layer, forming a photoresist layer on the hardmask layer, exposing and developing the photoresist layer to form a photoresist pattern, selectively removing the hardmask layer using the photoresist pattern to expose a part of the material layer, and etching the exposed part of the material layer.

The substrate may be, e.g., a silicon wafer, a glass substrate, or a polymer substrate. The material layer may be a material to be finally patterned, e.g., a metal layer such as an aluminum layer or a copper layer, a semiconductor layer such as a silicon layer, or an insulation layer such as a silicon oxide layer or a silicon nitride layer. The material layer may be formed through a method such as a chemical vapor deposition (CVD) process.

The hardmask composition may be the same as described above, and may be applied by spin-on coating in a form of a solution. In an implementation, an application thickness of the hardmask composition may be, e.g., about 50 Å to about 200,000 Å.

The heat-treating of the hardmask composition may be performed, e.g., at about 100° C. to about 1,000° C. for about 10 seconds to about 1 hour. In an implementation, the heat-treating of the hardmask composition may include a plurality of heat-treating processes, e.g., a first heat-treating process, and a second heat-treating process.

In an embodiment, the heat-treating of the hardmask composition may include, e.g., one heat-treating process performed at about 100° C. to about 1,000° C. for about 10 seconds to about 1 hour. In an implementation, the heat-treating may be performed under an atmosphere of air or nitrogen, or an atmosphere having oxygen concentration of 1 wt % or less.

In an implementation, the heat-treating of the hardmask composition may include, e.g., a first heat-treating process performed at about 100° C. to about 1,000° C., about 100° C. to about 800° C., about 100° C. to about 500° C., or about 150° C. to about 400° C. for, e.g., about 30 seconds to about 1 hour, about 30 seconds to about 30 minutes, about 30 seconds to about 10 minutes, about 30 seconds to about 5 minutes.

In an implementation, the heat-treating of the hardmask composition may include, e.g., a second heat-treating process performed at about 100° C. to 1,000° C., about 300° C. to about 1,000° C., about 500° C. to about 1,000° C., about 500° C. to about 600° C. for, e.g., about 30 seconds to about 1 hour, about 30 seconds to about 30 minutes, about 30 seconds to about 10 minutes, about 30 seconds to about 5 minutes, consecutively. In an implementation, the first and second heat-treating process may be performed under an air or nitrogen atmosphere, or may be performed in an atmosphere with an oxygen concentration of 1 wt % or less.

By performing at least one of the steps of heat-treating the hardmask composition at a high temperature, e.g., of 200° C. or higher, high etch resistance capable of withstanding etching gas and chemical liquid exposed in subsequent processes including the etching process may be exhibited.

In an implementation, the forming of the hardmask layer may include a UV/Vis curing process or a near IR curing process.

In an implementation, the forming of the hardmask layer may include a first heat-treating process, a second heat-treating process, a UV/Vis curing process, or a near IR curing process, or may include two or more processes consecutively.

In an implementation, the method may further include forming a silicon-containing thin layer on the hardmask layer. The silicon-containing thin layer may be formed of, e.g., SiCN, SiOC, SiON, SiOCN, SiC, SiO, SiN, or the like.

In an implementation, the method may further include forming a bottom antireflective coating (BARC) on the silicon-containing thin layer or on the hardmask layer before forming the photoresist layer.

In an implementation, exposure of the photoresist layer may be performed using, e.g., ArF, KrF, or EUV. After exposure, heat-treating may be performed at about 100° C. to about 700° C.

In an implementation, the etching process of the exposed part of the material layer may be performed through a dry etching process using an etching gas and the etching gas may be, e.g., N₂/O₂, CHF₃, CF₄, Cl₂, BCl₃, or a mixed gas thereof.

The etched material layer may be formed in a plurality of patterns, and the plurality of patterns may include a metal pattern, a semiconductor pattern, an insulation pattern, or the like, e.g., diverse patterns of a semiconductor integrated circuit device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Synthesis of Compounds

### Synthesis Example 1

### 1st Process: Friedel-Craft Acylation Reaction

9.48 g (0.05 mol) of pyrene, 25.00 g (0.09 mol) of pyrene carboxylic acid chloride, and 237.92 g of 1,2-dichloroethane were added to a flask to prepare a solution. 25.00 g (0.19 mol) of aluminum chloride was slowly added to the solution and then, stirred at ambient temperature for 10 hours. When a reaction was completed, methanol was added thereto, and precipitates formed therein were filtered to obtain bis(pyrenylcarbonyl) pyrene.

### 2nd Process: Grignard Reaction

30.00 g (0.05 mol) of the obtained bis(pyrenylcarbonyl) pyrene and 235.73 g of tetrahydrofuran were added to a flask to prepare a solution. Subsequently, 21.74 g (0.18 mol) of methylmagnesium bromide was slowly added to the solution at ambient temperature and then, stirred at 60° C. for 4 hours. When a reaction was completed, the resultant was neutralized to about pH 7 by using a 1% hydrogen chloride aqueous solution. Subsequently, Compound A represented by Chemical Formula 5 was obtained through extraction with ethyl acetate.

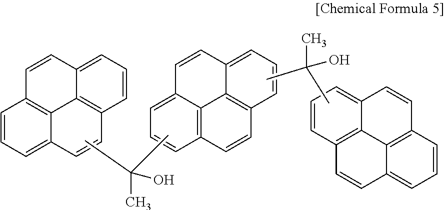

### Synthesis Example 2

Compound B represented by Chemical Formula 6 was obtained in the same manner as in Synthesis Example 1 except that 26.83 g (0.18 mol) of propylmagnesium bromide was used instead of 21.74 g (0.18 mol) of the methylmagnesium bromide in the second process.

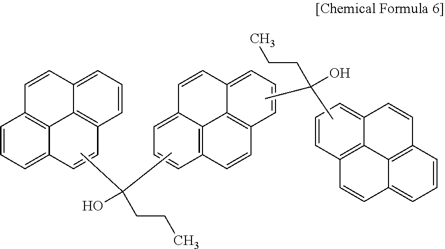

### Synthesis Example 3

### 1 st Process: Friedel-Craft Acylation Reaction

10.73 g (0.05 mol) of 4-methoxypyrene, 24.76 g (0.09 mol) of pyrene carboxylic acid chloride, and 239.88 g of 1,2-dichloroethane were added to a flask to prepare a solution. 24.63 g (0.18 mol) of aluminum chloride was slowly added to the solution and then, stirred at ambient temperature for 4 hours. When a reaction was completed, methanol was added thereto, and precipitates formed therein were filtered to obtain bis(pyrenylcarbonyl) methoxypyrene.

### 2nd Process: Demethylation Reaction

28.00 g (0.04 mol) of the obtained bis(pyrenylcarbonyl) methoxypyrene, 24.68 g (0.12 mol) of 1-dodecanethiol, 9.12 g (0.16 mol) of potassium hydroxide, and 247.20 g of N,N-dimethyl formamide were added to a flask and then, stirred at 90° C. for 6 hours. After the stirring, the resultant was cooled and then, neutralized to about pH 7 with a 1% hydrogen chloride aqueous solution, and precipitates formed therein were filtered to obtain bis(pyrenylcarbonyl) hydroxypyrene.

### 3rd Process: Grignard Reaction

26.02 g (0.04 mol) of the obtained bis(pyrenylcarbonyl) hydroxypyrene and 240.00 g of tetrahydrofuran were added to a flask to prepare a solution. 33.96 g (0.23 mol) of propylmagnesium bromide was slowly added thereto at ambient temperature and then, heated to 60° C. and stirred for 12 hours. When a reaction was completed, the resultant was neutralized to about pH 7 by using a 1% hydrogen chloride aqueous solution. Subsequently, Compound C represented by Chemical Formula 7 was obtained through extraction with ethyl acetate.

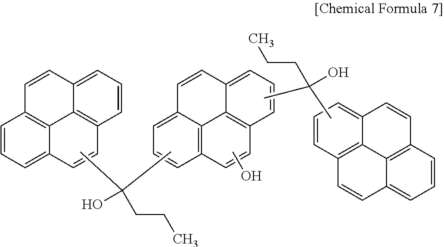

### Synthesis Example 4

Compound D represented by Chemical Formula 8 was obtained in the same manner as in Synthesis Example 1 except that 26.83 g (0.18 mol) of isopropylmagnesium bromide was used instead of 21.74 g (0.18 mol) of the methylmagnesium bromide in the second process.

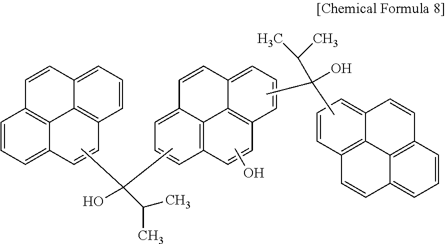

### Synthesis Example 5

### 1st Process: Friedel-Craft Acylation Reaction

20.60 g (0.10 mol) of terephthaloyl chloride, 47.00 g (0.20 mol) of 4-methoxypyrene, and 221.00 g of 1,2-dichloroethane were added to a flask to prepare a solution. 27.00 g (0.20 mol) of aluminum chloride was slowly added thereto and then, stirred at ambient temperature at 60° C. for 8 hours. When a reaction was completed, methanol was added to the solution, and precipitates formed therein were filtered to obtain bis(methoxypyrenylcarbonyl)benzene.

### 2nd Process: Demethylation Reaction

53.50 g (0.09 mol) of bis(methoxypyrenylcarbonyl)benzene, 91.10 g (0.45 mol) of 1-dodecanethiol, 30.30 g (0.54 mol) of potassium hydroxide, and 262.00 g of N,N-dimethyl formamide were added to a flask and then, stirred at 120° C. for 8 hours. After the stirring, the resultant was cooled and then, neutralized to about pH 7 by using a 1% hydrogen chloride aqueous solution, and precipitates formed therein were filtered to obtain bis(hydroxypyrenylcarbonyl) benzene.

### 3rd Process: Grignard Reaction

18.44 g (0.03 mol) of the obtained bis(hydroxypyrenylcarbonyl) benzene and 258.87 g of tetrahydrofuran were added to a flask to prepare a solution. 38.89 g (0.26 mol) of propylmagnesium bromide was slowly added thereto at ambient temperature and then, heated to 60° C. and stirred for 12 hours. When a reaction was completed, the resultant was neutralized to about pH 7 by using a 1% hydrogen chloride an aqueous solution, and Compound E represented by Chemical Formula 9-1 was obtained through extraction with ethyl acetate.

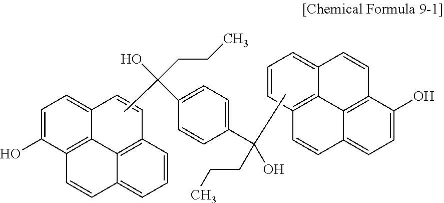

### Synthesis Example 6

### 1st Process: Friedel-Craft Acylation Reaction

50.00 g (0.17 mol) of coronene, 56.80 g (0.34 mol) of 4-methoxybenzochloride, and 353.00 g of 1,2-dichloroethane were added to a flask to prepare a solution. 44.4 g (0.34 mol) of aluminum chloride was slowly added to the solution at ambient temperature and then, heated to 60° C. and stirred for 8 hours. When a reaction was completed, methanol was added thereto, and precipitates formed therein were filtered to obtain bis(methoxybenzoyl) coronene.

### 2nd Process: Demethylation Reaction

50.00 g (0.88 mol) of the obtained bis(methoxybenzoyl) coronene, 89.00 g (0.44 mol) of 1-dodecanethiol, 29.60 g (0.53 mol) of potassium hydroxide, and 253.00 g of N,N-dimethyl formamide were added to a flask and then, stirred at 120° C. for 8 hours. After the stirring, the resultant was cooled and then, neutralized to about pH 7 by using a 1% hydrogen chloride aqueous solution, and precipitates formed therein were filtered to obtain bis(hydroxybenzoyl) coronene.

### 3rd Process: Grignard Reaction

17.86 g (0.03 mol) of the obtained bis(hydroxybenzoyl) coronene and 258.87 g of tetrahydrofuran were added to a flask to prepare a solution. 38.97 g (0.26 mol) of propylmagnesium bromide was slowly added thereto at ambient temperature and then, heated to 60° C. for 12 hours. When a reaction was completed, the resultant was neutralized to about pH 7 by using a 1% hydrogen chloride aqueous solution, and Compound F represented by Chemical Formula 10-1 was obtained through extraction with ethyl acetate.

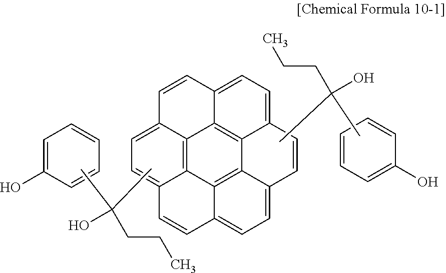

### Comparative Synthesis Example

### 1 st Process: Friedel-Craft Acylation Reaction

50.0 g (0.166 mol) of coronene, 56.8 g (0.333 mol) of 4-methoxybenzoyl chloride, and 353 g of 1,2-dichloroethane were added to a flask to prepare a solution. 44.4 g (0.333 mol) of aluminum chloride was slowly added to the solution at ambient temperature and then, stirred at 60° C. for 8 hours. When a reaction was completed, methanol was added thereto, and precipitates formed therein were filtered to obtain double-substituted 4-methoxybenzoyl coronene.

### 2nd Process: Demethylation Reaction

50.0 g (0.880 mol) of the obtained double-substituted 4-methoxybenzoyl coronene, 89.0 g (0.440 mol) of 1-dodecanethiol, 29.6 g (0.528 mol) of potassium hydroxide, and 53 g of N,N-dimethylformamide were added to a flask and then, stirred at 120° C. for 8 hours. After the stirring, the resultant was cooled, neutralized to about pH 7 by using a 10% hydrogen chloride aqueous solution, and then, extracted with ethyl acetate to obtain double-substituted 4-hydroxybenzoyl coronene.

### 3rd Process: Reduction Reaction

25.0 g (0.0463 mol) of the obtained double substituted 4-hydroxybenzoyl group coronene and 170 g of tetrahydrofuran were added to a flask to prepare a solution. 17.5 g (0.463 mol) of a sodium borohydride aqueous solution was slowly added thereto and then, stirred for 24 hours at ambient temperature. When a reaction was completed, the resultant was neutralized to about pH 7 by using a 10% hydrogen chloride aqueous solution and then, extracted with ethyl acetate to obtain Compound G represented by Chemical Formula 11.

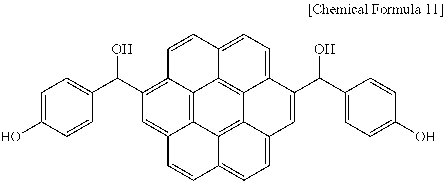 [SMILES: Oc1ccc(C(O)c2cc3ccc4ccc5cc(C(O)c6ccc(O)cc6)c6ccc7ccc2c2c3c4c5c6c72)cc1]

### Preparation of Hardmask Composition

### Examples and Comparative Examples

Each of Compounds A to G according to Synthesis Examples 1 to 6 and the Comparative Synthesis Example were uniformly dissolved in a mixed solvent of propylene glycol monomethyl ether acetate (PGMEA) and cyclohexanone in a volume ratio of 3:7 to prepare a hardmask composition having a compound content of 10.0 wt %.

### Evaluation 1: Heat Resistance Evaluation

Each of the hardmask compositions of Examples 1 to 6 and the Comparative Example were spin-coated on a silicon wafer and then, heat-treated on a hot plate at 160° C. for 1 minute to form a hardmask layer. A thickness of the hardmask layer was measured by using a film thickness meter made by K-MAC Co., Ltd. Subsequently, the hardmask layer was reheat-treated at 400° C. for 2 minutes to remeasure a thickness of the hardmask layer. When the hardmask layer had a thickness variation ratio of less than 5% before and after the reheat-treatment at 400° C. for 2 minutes, an “A” (very good) was given, when the thickness variation ratio was greater than or equal to 5% and less than 10%, a “B” (good) was given, and when the thickness variation ratio was greater than or equal to 10%, a “C” (inferior) was given. The results are shown in Table 1.

TABLE 1

| | Heat resistance |
|---|---|
| Example 1 | A |
| Example 2 | A |
| Example 3 | A |
| Example 4 | A |
| Example 5 | A |
| Example 6 | A |
| Comparative Example | C |

Referring to Table 1, the hardmask layers formed of the compositions of Examples 1 to 6 exhibited a thickness variation ratio of less than 5% before and after the reheat treatment, and the hardmask layer formed of the composition according to the Comparative Example exhibited a thickness variation ratio of greater than or equal to 10% before and after the reheat treatment. In other words, the hardmask layers formed of the compositions of Examples 1 to 6 exhibited excellent heat resistance, compared to the hardmask layer formed of the composition according to the Comparative Example.

### Evaluation 2: Gap-Fill Characteristics

Each of the hardmask compositions Examples 1 to 6 and the Comparative Example were spin-coated on a patterned silicon wafer and then, heat-treated at 400° C. for 60 seconds to form a hardmask layer. A pattern cross-section image of the hardmask layer was examined with a field emission scanning electron microscope (FE-SEM) to check whether or not voids occurred. The results are shown in Table 2.

TABLE 2

| | Whether voids occur or not |
|---|---|
| Example 1 | Voids did not occur |
| Example 2 | Voids did not occur |
| Example 3 | Voids did not occur |
| Example 4 | Voids did not occur |
| Example 5 | Voids did not occur |
| Example 6 | Voids did not occur |
| Comparative Example | Voids occurred |

Referring to Table 2, the hardmask layers formed of the hardmask compositions of Examples 1 to 6 did not exhibit voids, but the hardmask layer formed of the hardmask composition of the Comparative Example did exhibit voids. Accordingly, the hardmask layers formed of the hardmask compositions of Examples 1 to 6 exhibited more excellent gap-fill characteristics, compared with the hardmask layer formed of the hardmask composition of the Comparative Example.

### Evaluation 3: Planarization Characteristics

Each of the hardmask compositions of Examples 1 to 6 and the Comparative Example were respectively spin-coated on a patterned silicon wafer and heat-treated at 400° C. for 60 seconds, and a pattern cross-section image thereof was examined through a field emission scanning electron microscope (FE-SEM). A thickness of the hardmask layer shown in the pattern cross-section image was measured, which was used to digitalize planarization degrees according to Calculation Equation 1 of the FIGURE. Ratios of the planarization degrees of the hardmask layer formed of each of the compositions according to Examples 1 to 6 to the planarization degrees of the hardmask layer formed of the composition according to the Comparative Example were calculated to evaluate planarization characteristics. If the ratio was less than 5% an “A” (very good) was given, if the ratio was greater than or equal to 5% and less than 10%, a “B” (good) was given, and if the ratio was greater than or equal to 10%, a “C” (inferior) was given. The results are shown in Table 3.

TABLE 3

| | Planarization characteristics |
|---|---|
| Example 1 | A |
| Example 2 | A |
| Example 3 | A |
| Example 4 | A |
| Example 5 | A |
| Example 6 | A |
| Comparative Example | — |

### Evaluation 4: Etch Resistance

Each of the hardmask compositions of Examples 1 to 6 and the Comparative Example were spin-coated on a silicon wafer and then, heat-treated on a hot plate at 160° C. for 1 minute to form a hardmask layer. The hardmask layers were dry etched by using CF₄/Ar mixed gas for 30 seconds and then, measured with respect to a hardmask thickness before and after the etching by using a film thickness meter made by K-MAC Co., Ltd. to calculate an etch rate. Herein, etch resistance was evaluated by using a ratio of the etch rate of the hardmask layer formed of each of the compositions according to Examples 1 to 6 to the etch rate of the hardmask layer formed of the composition according to the Comparative Example. If the ratio was less than 0.95, “A” (very good) was given, greater than or equal to 0.95 and less than 1.00, “B” (good) was given, and greater than or equal to 1.00, “C” (inferior) was given. The results are shown in Table 4.

TABLE 4

| | Etch resistance |
|---|---|
| Example 1 | A |
| Example 2 | A |
| Example 3 | A |
| Example 4 | A |
| Example 5 | A |
| Example 6 | A |
| Comparative Example | — |

Referring to Table 4, the hardmask layer formed of the composition of the Comparative Example exhibited an etch rate of 0.95, compared with that of the hardmask layer formed of each of the compositions of Examples 1 to 6. Accordingly, the hardmask layers formed of the compositions of Examples 1 to 6 exhibited excellent etch resistance, compared with the hardmask layer formed of the composition of the Comparative Example.

By way of summation and review, there is a constant trend in a semiconductor industry to reduce a size of chips. In order to respond to this, the line width of the photoresist pattern in lithography technology must have a size of several tens of nanometers. Therefore, a height that can withstand the line width of the photoresist pattern may be limited, and there are cases where the photoresists do not have sufficient resistance in the etching step. In order to compensate for this, an auxiliary layer, which may be called a hardmask layer, may be used between a material layer to be etched and a photoresist layer. This hardmask layer may serve as an interlayer that transfers a fine pattern of the photoresist layer through selective etching. Therefore, the hardmask layer may be required to have sufficient etch resistance as to withstand the etching process during the pattern transfer.

Some hardmask layers may be formed in a chemical or physical deposition method and may have low economic efficiency due to large-scale equipment and a high process cost. Accordingly, a spin-coating technique for forming a hardmask layer has recently been developed. The spin-coating technique may be an easier process to conduct than some other methods, and a hardmask layer formed therefrom may exhibit excellent gap-fill characteristics and planarization characteristics. However, in the hardmask layer formed using the spin-coating technique the required etch resistance may be somewhat lowered. Accordingly, a hardmask composition may be applied using the spin-coating technique to secure equivalent etch resistance to that of the hardmask layer formed in the chemical or physical deposit method.

Accordingly, in order to improve the etch resistance of a hardmask layer, research on maximizing carbon content of the hardmask composition is being made. However, as a carbon content of a compound included in the hardmask composition is maximized, solubility of the compound in a solvent may be reduced. Therefore, a carbon content of the compound included in the hardmask composition should be maximized to improve the etch resistance of the hardmask layer formed therefrom, while the compound must be soluble in the solvent.

The hardmask composition according to some embodiments may include a compound having an aromatic hydrocarbon ring, thereby increasing the carbon content in the composition. Accordingly, the hardmask layer obtained from the composition may secure excellent etch resistance. In addition, the compound may contain a specific functional group, thereby having a high carbon content and excellent solubility in solvents.

One or more embodiments may provide a hardmask composition that can be effectively applied to a hardmask layer.

One or more embodiments may provide a hardmask layer including a cured product of the hardmask composition.

One or more embodiments may provide a method of forming patterns using the hardmask composition.

The hardmask composition according to some embodiments has excellent solubility in solvents and can be effectively applied to a hardmask layer.

A hardmask layer formed from the hardmask composition according to some embodiments can secure excellent heat resistance and excellent etch resistance.

A hardmask layer formed from the hardmask composition according to some embodiments can secure excellent planarization characteristics.

The hardmask composition according to some embodiments may have excellent solubility in solvents and may be effectively applied to a hardmask layer.

A hardmask layer formed from the hardmask composition according to some embodiments may secure excellent heat resistance and excellent etch resistance.

A hardmask layer formed from the hardmask composition according to some embodiments may secure excellent planarization characteristics.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A hardmask composition, comprising:
a compound represented by Chemical Formula 1;
and a solvent, 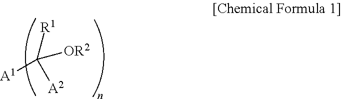
wherein, in Chemical Formula 1,
A¹ and A² are each independently a substituted or unsubstituted C6 to C30 aromatic hydrocarbon group, or a group in which substituted or unsubstituted C6 to C30 aromatic hydrocarbon groups are linked by a single bond, a substituted or unsubstituted C1 to C5 alkylene group, or a combination thereof,
R¹ is a substituted or unsubstituted monovalent C1 to C30 saturated aliphatic hydrocarbon group, a substituted or unsubstituted monovalent C2 to C30 unsaturated aliphatic hydrocarbon group, a substituted or unsubstituted monovalent C3 to C30 saturated or unsaturated alicyclic hydrocarbon group, or a substituted or unsubstituted C6 to C30 aromatic hydrocarbon group,
R² is hydrogen, deuterium, or a substituted or unsubstituted C1 to C20 alkyl group,
n is an integer of 1 to 6, and
if n is an integer of 2 or more, each of R¹, R², and A² is the same or different from each other.

2. The hardmask composition as claimed in claim 1, wherein A¹ and A² of Chemical Formula 1 are each independently a substituted or unsubstituted group of a moiety of Group 1:

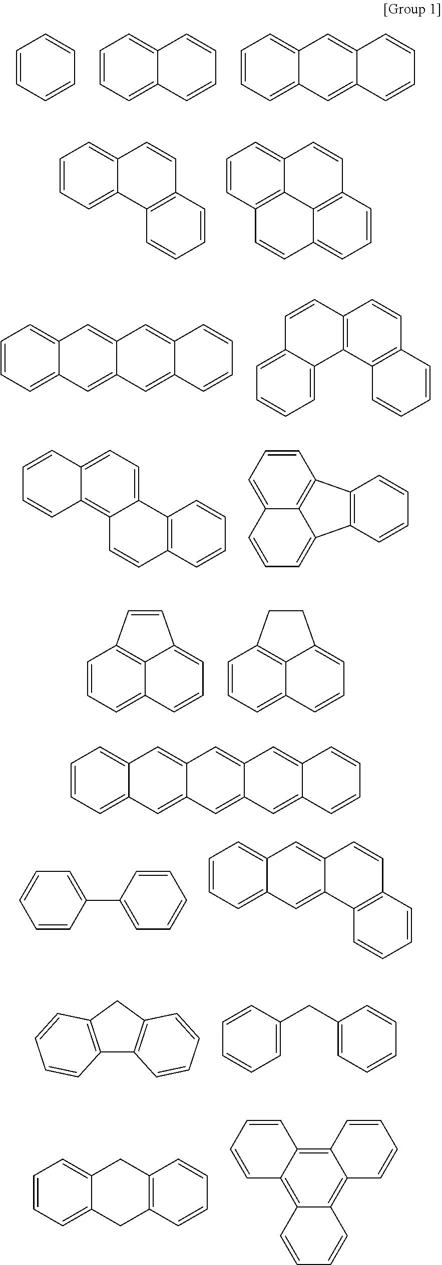

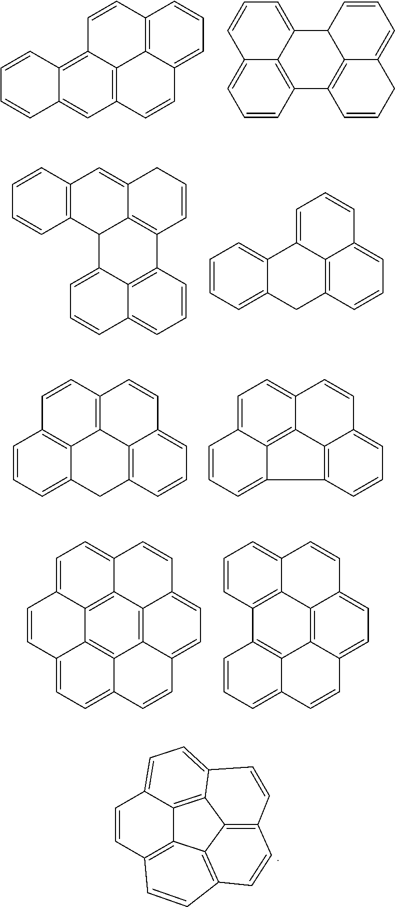

3. The hardmask composition as claimed in claim 1, wherein A¹ and A³ of Chemical Formula 1 are each independently a substituted or unsubstituted group of a moiety of Group 1-1:

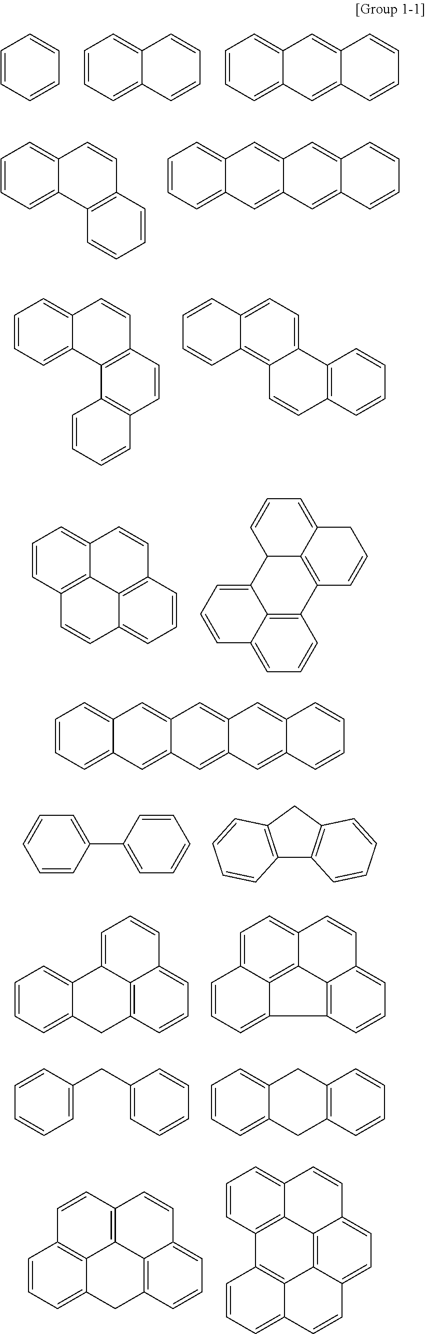

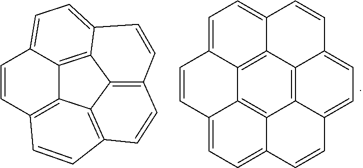

4. The hardmask composition as claimed in claim 1, wherein A¹ in Chemical Formula 1 is a substituted or unsubstituted group of a moiety of Group 1-2:

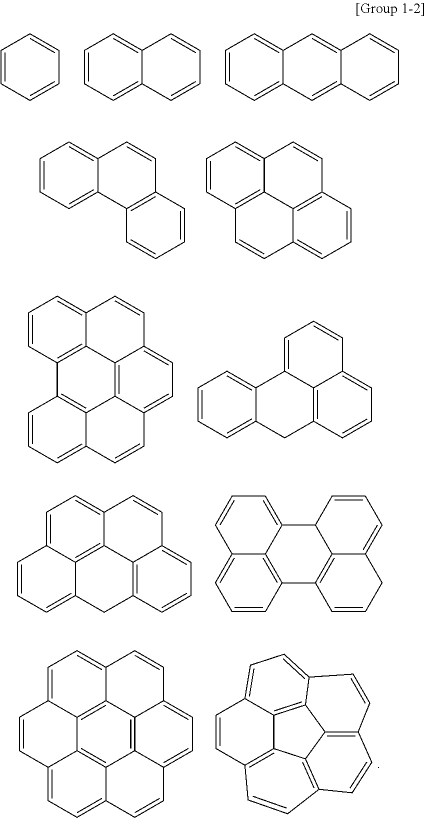

5. The hardmask composition as claimed in claim 1, wherein A² of Chemical Formula 1 is a substituted or unsubstituted group of a moiety of Group 1-3:

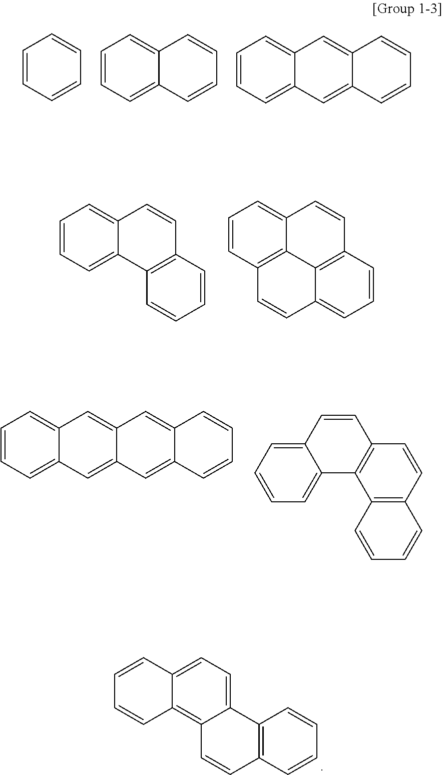

6. The hardmask composition as claimed in claim 1, wherein R¹ in Chemical Formula 1 is a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group.

7. The hardmask composition as claimed in claim 1, wherein R² in Chemical Formula 1 is hydrogen, deuterium, a substituted or unsubstituted methyl group, a substituted or unsubstituted ethyl group, a substituted or unsubstituted propyl group, a substituted or unsubstituted butyl group, or a substituted or unsubstituted pentyl group.

8. The hardmask composition as claimed in claim 1, wherein, in Chemical Formula 1,
R¹ is a substituted or unsubstituted C1 to C20 alkyl group or a substituted or unsubstituted C6 to C20 aryl group,
R² is hydrogen, deuterium, a substituted or unsubstituted methyl group, or a substituted or unsubstituted ethyl group, and
n is an integer of 1 to 3.

9. The hardmask composition as claimed in claim 7, wherein, in Chemical Formula 1,
n is 2 or 3, and
R¹, R², and A² are the same.

10. The hardmask composition as claimed in claim 1, wherein, in Chemical Formula 1,
R¹ is a substituted or unsubstituted C1 to C10 alkyl group,
R² is hydrogen, or deuterium, and
n is 2.

11. The hardmask composition as claimed in claim 1, wherein:
the compound represented by Chemical Formula 1 is represented by one of Chemical Formula 2 to Chemical Formula 4: 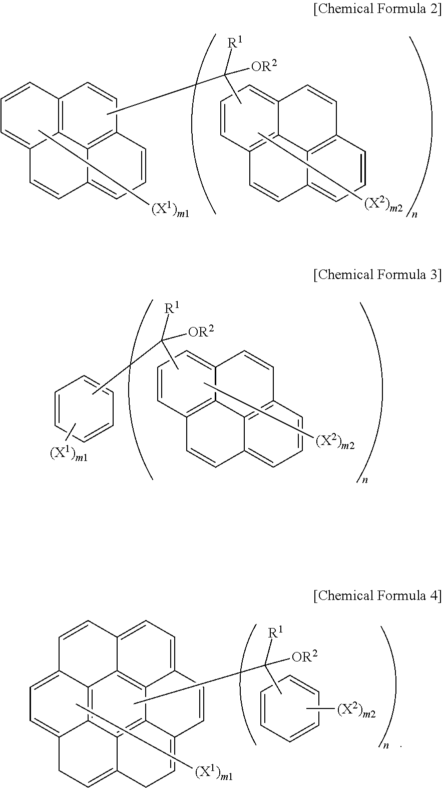
in Chemical Formula 2 to Chemical Formula 4,
R¹ is a substituted or unsubstituted C1 to C20 alkyl group, a substituted or unsubstituted C2 to C20 alkenyl group, a substituted or unsubstituted C3 to C20 cycloalkyl group, or a substituted or unsubstituted C6 to C20 aryl group,
R² is hydrogen, deuterium, or a substituted or unsubstituted C1 to C10 alkyl group,
X¹ and X² are each independently, deuterium, a hydroxy group, a halogen atom, a thiol group, a substituted or unsubstituted C1 to C10 alkoxy group, or a substituted or unsubstituted C1 to C10 alkyl group,
n is an integer of 1 to 3, and
m1 and m2 are each independently an integer of 0 to 5.

12. The hardmask composition as claimed in claim 1, wherein:
the compound represented by Chemical Formula 1 is represented by one of Chemical Formula 5 to Chemical Formula 10,

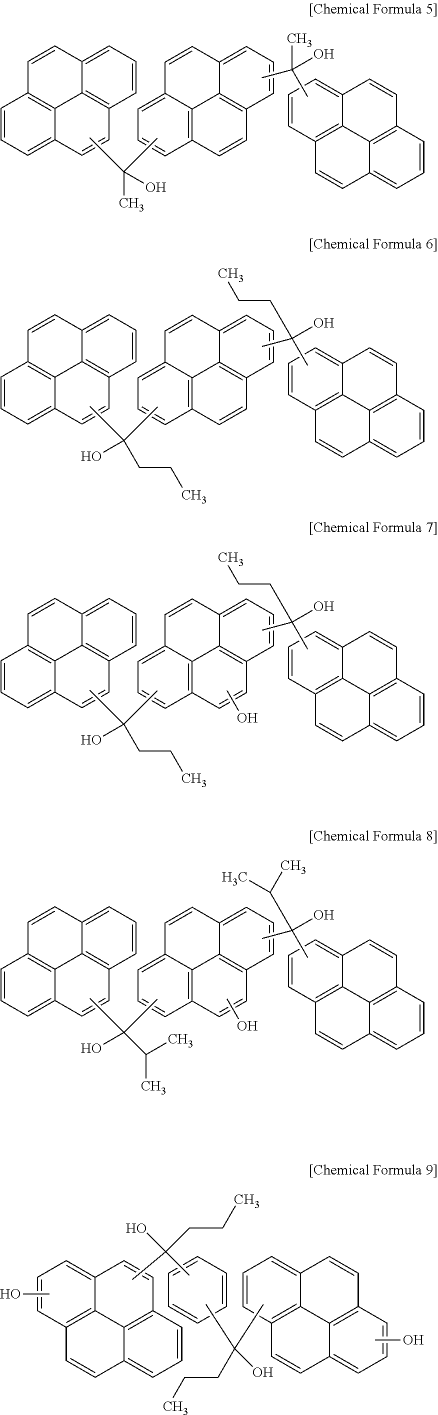

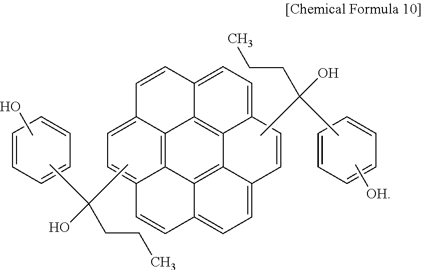

13. The hardmask composition as claimed in claim 1, wherein the compound has a molecular weight of about 500 g/mol to about 10,000 g/mol.

14. The hardmask composition as claimed in claim 1, wherein the compound is included in an amount of about 0.1 wt % to about 30 wt %, based on a total weight of the hardmask composition.

15. The hardmask composition as claimed in claim 1, wherein the solvent includes propylene glycol, propylene glycol diacetate, methoxy propanediol, diethylene glycol, diethylene glycol butylether, tri (ethylene glycol) monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, methylpyrrolidinone, acetylacetone, or ethyl 3-ethoxypropionate.

16. A hardmask layer comprising a cured product of the hardmask composition as claimed in claim 1.

17. A method of forming patterns, the method comprising:
providing a material layer on a substrate;
applying the hardmask composition as claimed in claim 1 to the material layer;
heat-treating the hardmask composition to form a hardmask layer;
forming a photoresist layer on the hardmask layer;
exposing and developing the photoresist layer to form a photoresist pattern;
selectively removing the hardmask layer using the photoresist pattern to expose a portion of the material layer; and
etching an exposed part of the material layer.

18. The method as claimed in claim 17, wherein forming the hardmask layer includes heat-treating at about 100° C. to about 1,000° C.
